(19) United States
(12) Patent Application Publication
(10) Pub. No.: US 2012/0251496 A1
(43) Pub. Date: Oct. 4, 2012

(54) EZATIOSTAT FOR TREATING MULTIPLE MYELOMA

(75) Inventor: Michael M. Wick, Palo Alto, CA (US)

(73) Assignee: Telik, Inc.

(21) Appl. No.: 13/425,234

(22) Filed: Mar. 20, 2012

Related U.S. Application Data
(60) Provisional application No. 61/470,357, filed on Mar. 31, 2011.

(51) Int. Cl. A61K 31/216; A61K 31/664; A61K 31/573; A61K 35/66; A61K 38/21; A61K 31/454; A61K 31/4745; A61P 35/00; A61K 31/69; A61K 31/704

(52) U.S. Cl. 424/85.7; 514/533; 514/64; 514/110; 514/171; 514/34; 514/323; 514/283; 424/93.7

(57) ABSTRACT

Ezatiostat is useful for inhibiting multiple myeloma cell proliferation and treating multiple myeloma, alone or when added together with another anti-myeloma drug.

### CROSS-REFERENCE TO RELATED APPLICATION

This application claims the priority under 35 U.S.C. 119(e) of U.S. Provisional Application No. 61/470,357, filed Mar. 31, 2011, the entire disclosure of which is incorporated herein by reference.

### BACKGROUND OF THE INVENTION

1. Field of Invention

This invention provides methods and compositions for treating multiple myeloma with ezatiostat administered alone or in combination with another drug and/or therapy.

2. State of the Art

Multiple myeloma is a hematologic malignancy characterized by the proliferation of a single clone of plasma cells engaged in the production of an immunoglobulin. Bone pain, anemia, and fatigue constitute some of the symptoms of multiple myeloma. Hypercalcemia and renal insufficiency are also manifestations of this malignancy.

Conditions associated with a diagnosis of multiple myeloma include bone marrows with greater than 10% plasma cells or plasmacytoma coupled with one or more of the following: monoclonal protein in serum (usually greater than 3 g/deciliter (dL)), monoclonal protein in urine, and lytic bone lesions. Multiple myeloma accounts for more than 10% of hematologic malignancies with the incidence of approximately of 1 to 4 individuals per 100,000 per year. For multiple myeloma, the median age at diagnosis is 61; the advanced age itself limits the types of treatment the patient can undergo.

Chemotherapy is the preferred initial treatment for multiple myeloma. Yet, for chemotherapy alone, the 5 year survival probability is only 12%. Almost all patients with multiple myeloma who respond positively to chemotherapy will eventually experience relapse. Patients relapse in a large part because their multiple myeloma becomes resistant (refractory) to the initial treatment, and such patients then are treated with a drug cocktail. For example, patients with multiple myeloma resistant to alkylating agents are next treated with a drug cocktail regimen called VAC (vincristine, doxorubicin or adriamycin, and dexamethasone).

As part of a chemotherapy regimen, thalidomide has been evaluated in relapsed/refractory myeloma patients, though thalidomide combinations with chemotherapy, specifically anthracyclines, carry an increased risk of venous thromboembolic (VTE) complications, which often require extensive patient monitoring and intense prophylaxis. Thalidomide alone produced partial response rates in about 30% of patients. The overall response rate can be enhanced when thalidomide is administered as part of a drug cocktail; however, the improvement in response rate comes with an increase in the undesirable side effects, such as VTE, as discussed above.

Bortezomib is a proteasome inhibitor with antimyeloma activity as a single agent, though bortezomib-induced peripheral neuropathy remains a dose-limiting toxicity in patients with multiple myeloma, which often requires adjustment of treatment and affects quality of life.

Lenalidomide is an agent approved recently for relapse/refractory myeloma in the United States and Europe. However, as with its structural analog thalidomide, lenalidomide is associated with adverse effects related to VTE. Nearly all lenalidomide trials show an increased risk of VTE with lenalidomide therapy, with the risk significantly increasing when lenalidomide is combined with dexamethasone or with other combination chemotherapy.

Autologous peripheral stem cell transplantation is useful for up to 50% of multiple myeloma patients. Despite a low mortality rate, problems with such transplant therapy include the inability to eradicate the tumor and the difficulty in the removal of myeloma cells and their precursors from the stem cell collection used for transplantation.

Allogenic transplant is another therapy option for treating multiple myeloma, but is less frequently used since the mortality rate at 100 days is 25-30% and it does not provide a cure. Only 5-10% of patients with multiple myeloma are eligible for allogeneic bone marrow transplantation because of their age and the paucity of a human leukocyte antigen (HLA)-matched sibling donor.

Use of allogenic transplant for the treatment of relapsed myeloma also remains a treatment strategy with limited clinical benefit. Most studies evaluating its use in this setting demonstrate long-term disease-free survival of 10-20%, with a significant fraction of patients developing debilitating chronic graft versus host disease or relapse. Given the significant limitations of treatment-related mortality, morbidity, and poor overall outcomes, the use of allogenic transplant for the management of relapsed myeloma is deemed not effective. There is a need for a new treatment regimen for multiple myeloma.

### SUMMARY OF THE INVENTION

In one of its method aspects, the present invention provides a method of treating a multiple myeloma tumor which method comprises contacting said tumor with an effective amount of ezatiostat. In another of its method aspects, the present invention provides a method of killing a multiple myeloma cell comprising contacting the multiple myeloma cells with an effective amount of ezatiostat. In another of its method aspects, the present invention provides a method of treating multiple myeloma in an afflicted patient which method comprises administering to said patient a therapeutically effective amount of a composition comprising ezatiostat. In various embodiments, the method aspects further comprise contacting or administering in combination or coadministration of one or more additional drugs to treat multiple myeloma.

Administration in “combination” or “coadministration” refers to the administration of the two or more agents (e.g., ezatiostat and one or more additional drugs to treat multiple myeloma) in any manner in which the pharmacological effects of both are manifest in the patient at the same time. Thus, administration in combination does not require that a single pharmaceutical composition, the same dosage form, or even the same route of administration be used for administration of both ezatiostat and the additional drug(s) to treat multiple myeloma, or that the agents be administered at precisely the same time. However, administration in combination will be accomplished most conveniently by the same dosage form and the same route of administration, at substantially the same time. Obviously, such administration most advantageously proceeds by delivering both active ingredients simultaneously in a novel pharmaceutical composition as provided herein below.

In one of its composition aspects, the present invention provides a composition comprising an effective amount of ezatiostat to treat multiple myeloma and an effective amount of one or more additional drugs to treat multiple myeloma.

### BRIEF DESCRIPTION OF THE FIGURE

FIG. 1 graphically illustrates synergistic activity of ezatiostat hydrochloride added together with an additional drug for treating multiple myeloma, melphalan, in human multiple myeloma RPMI8226 cells.

### DETAILED DESCRIPTION OF THE INVENTION

As noted above, this invention is directed to methods and compositions for treating multiple myeloma by administering ezatiostat alone or in addition with other drugs and therapies. However, prior to discussing this invention in further detail, the following terms will be defined.

### DEFINITIONS

As used herein, the following terms have the following meanings

The singular forms “a,” “an,” and “the” and the like include plural referents unless the context clearly dictates otherwise. Thus, for example, reference to “a drug” includes both a single drug and a plurality of different drugs.

The term “about” when used before a numerical designation, e.g., temperature, time, amount, and concentration, including a range, indicates approximations which may vary by ±10%, ±5%, or ±1%.

“Administration” refers to introducing a drug, an agent, or a therapy to a patient. A therapeutic amount can be administered, which can be determined by the treating physician or the like. For drugs and agents, a parenteral route of administration is preferred. The related terms and phrases “administering” and “administration of”, when used in connection with a compound or pharmaceutical composition (and grammatical equivalents) refer both to direct administration, which may be administration to a patient by a medical professional and/or to indirect administration, which may be the act of prescribing a drug. For example, a physician who provides a patient with a prescription for a drug is administering the drug to the patient. In any event, administration entails delivery to the patient of the drug or an agent.

“Comprising” or “comprises” is intended to mean that the compositions and methods include the recited elements, but not exclude others. “Consisting essentially of” when used to define compositions and methods, shall mean excluding other elements of any essential significance to the combination for the stated purpose. Thus, a method consisting essentially of the elements as defined herein would not exclude other steps or composition that do not materially affect the basic and novel characteristic(s) of the claimed invention. “Consisting of” shall mean excluding more than trace elements of other ingredients and substantial method steps. Embodiments defined by each of these transition terms are within the scope of this invention.

“Effective amount” as used herein in the context of treating multiple myeloma tumor or killing multiple myeloma cells refers to an amount or a concentration (e.g., expressed in micro molar) that can kill at least about 10%, at least about 25%, at least about 50%, at least about 75%, at least about 90%, and at least about 99% of cells in a tumor or in a cell culture.

Ezatiostat refers to a compound of formula:

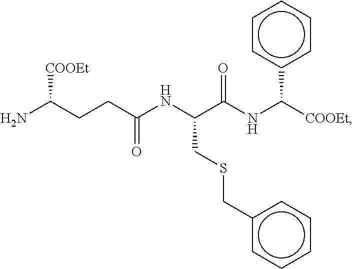

which is also known as TLK199 or TER 199. The term “ezatiostat” includes salts thereof, preferably a pharmaceutically acceptable salt thereof.

“Pharmaceutically acceptable salt” refers to acid addition salts of basic compounds, e.g., those compounds including a basic amino group, and to basic salts of acidic compounds, e.g., those compounds including a carboxyl group, and to amphoteric salts of compounds that include both an acidic and a basic moiety, such that these salts are suitable for administration in vivo, preferably to humans. Various organic and inorganic acids may be used for forming acid addition salts. Pharmaceutically acceptable salts are derived from a variety of organic and inorganic counter ions well known in the art. Pharmaceutically acceptable salts include, when the molecule contains a basic functionality, by way of example only, hydrochloride, hydrobromide, tartrate, mesylate, acetate, maleate, oxalate and the like, and when the molecule contains an acidic functionality, by way of example only, sodium, potassium, calcium, magnesium, ammonium, tetraalkylammonium, N-methylmorpholinium, and the like. In one embodiment, the pharmaceutically acceptable salt of ezatiostat is ezatiostat hydrochloride.

“Monotherapy” refers to administering a single active agent for treating a condition, such as multiple myeloma.

“Multiple myeloma” refers to a hematologic malignancy characterized by a proliferation of a single clone of plasma cells engaged in the production of an immunoglobulin. Bone pain, anemia, and fatigue constitute certain symptoms of multiple myeloma. Hypercalcemia and renal insufficiency are important manifestations of this hematologic malignancy. Conditions associated with a diagnosis of multiple myeloma include, without limitation, bone marrows with greater than 10% plasma cells or plasmacytoma coupled with one or more of the following: monoclonal protein in serum (usually greater than 3 g/deciliter (dL)), monoclonal protein in urine, and lytic bone lesions.

“Chemotherapy” as used in “multiple myeloma is refractory to one or more of chemotherapy” or “multiple myeloma relapsed after treatment with one or more of chemotherapy” refers to chemotherapy that includes, without limitation, monotherapy and combination or cocktail therapy involving additional drugs to treat multiple myeloma. Examples of such additional drugs to treat multiple myeloma include, without limitation, cyclophosphamide, dexamethasone, doxorubicin, interferon-alpha, melphalan, pegylated interferon-alpha, vincristine, and the like, corticosteroids, such as prednisone, dexamethasone, and the like, and immune modulating agents such as thalidomide, lenalidomide, and bortezomib, and the like.

“Patient” refers to a mammal and includes, a rat, a mouse, a dog, a horse, or a human patient.

“Primary refractory multiple myeloma” refers to multiple myeloma, which does not respond to induction or first line therapy.

“Relapsed and/or refractory multiple myeloma” refers to a multiple myeloma unresponsive to a drug or a therapy administered prior to treatment with ezatiostat. For example and without limitation, relapsed and/or refractory multiple myeloma includes multiple myeloma in patients whose first progression occurs in the absence of any treatment following successful treatment with a drug or a therapy; multiple myeloma in patients who progress on a treatment, or within 60 days of the treatment; and multiple myeloma in patients who progress while receiving treatment. Examples of relapsed and/or refractory multiple myeloma include, without limitation, bortezomib refractory relapse or lenalidomide refractory relapse multiple myeloma.

“Salvage therapy” refers to a form of treatment given after the multiple myeloma does not respond to first line or other subsequent treatment.

“Single active agent” refers to an agent that is useful for treating a condition, such as multiple myeloma, when administered without coadministration during the course of the treatment with the single active agent. In some embodiments, ezatiostat as a single active agent is contemplated to be effective in treating multiple myeloma, without coadministering a chemotherapy agent, stem cell transplantations, or radiation therapy.

“Therapeutically effective amount” or “therapeutic amount” refers to an amount of a drug or an agent that when administered to a patient suffering from a condition, will have the intended therapeutic effect, e.g., alleviation, amelioration, palliation or elimination of one or more symptoms or manifestations of the condition in the patient. For example, and without limitation, when the condition treated is multiple myeloma, the relevant symptoms or manifestations include, proliferation of a single clone of plasma cells engaged in the production of a specific immunoglobulin; a bone marrow with>10% plasma cells or plasmacytoma and one of the following: monoclonal protein in serum (usually>3 g/dL), monoclonal protein in urine, lytic bone lesions; bone pain; anemia; fatigue; hypercalcemia; and renal insufficiency. The therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, such as ezatiostat, in the context of treating multiple myeloma, refers to an amount of the agent that alleviates, ameliorates, palliates, or eliminates one or more manifestations of the multiple myeloma in the patient.

“Treatment”, “treating”, and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms or manifestations of the condition. Treatment, as used herein, also covers the treatment of multiple myeloma tumor in vivo, including in animals other than humans, ex vivo, and in vitro, e.g., in spheroids. For example, and without limitation, when the condition treated is multiple myeloma, the relevant symptoms or manifestations include, proliferation of multiple myeloma cells, bone pain, anemia, fatigue, hypercalcemia, and renal insufficiency. Symptoms of multiple myeloma also include those associated with smoldering myeloma, which is a slow-growing form of multiple myeloma.

### Methods and Compositions

In one of its method aspects, the present invention provides a method of treating a multiple myeloma tumor, which method comprises contacting said tumor with an effective amount of ezatiostat. In another of its method aspects, the present invention provides a method of killing a multiple myeloma cell comprising contacting the multiple myeloma cells with an effective amount of ezatiostat. In various embodiments, the methods further comprise contacting an additional drugs to treat multiple myeloma.

In another of its method aspects, the present invention provides a method of treating multiple myeloma in an afflicted patient which method comprises administering to said patient a therapeutically effective amount of a composition comprising ezatiostat. In various embodiments, the composition further comprises one or more additional drugs to treat multiple myeloma.

In another embodiment, the one or more additional drugs comprises bortezomib, cyclophosphamide, dexamethasone, doxorubicin, interferon-alpha, lenalidomide, melphalan, pegylated interferon-alpha, prednisone, thalidomide, or vincristine. In a preferred embodiment, the additional drug is lenalidomide.

In another embodiment, the therapeutically effective amount of said composition is administered as part of a first line (or induction) therapy.

In another embodiment, the therapeutically effective amount of said composition is administered as part of a salvage therapy in treating patients wherein the multiple myeloma has become refractory to other drugs for treating multiple myeloma. In another embodiment, the drug for treating multiple myeloma to which the multiple myeloma is refractory, includes, without limitation, bortezomib, cyclophosphamide, dexamethasone, doxorubicin, interferon-alpha, lenalidomide, melphalan, pegylated interferon-alpha, prednisone, thalidomide, and vincristine.

In another embodiment, the multiple myeloma is relapsed multiple myeloma. In another embodiment, the relapsed multiple myeloma relapsed after treatment with one or more of additional drugs to treat multiple myeloma, for example, and without limitation, bortezomib, cyclophosphamide, dexamethasone, doxorubicin, interferon-alpha, lenalidomide, melphalan, pegylated interferon-alpha, prednisone, thalidomide, and vincristine.

In another embodiment, the methods of treating multiple myeloma further comprise administering one or more additional therapies. In another embodiment, the additional therapy comprises a stem cell transplant therapy. In another embodiment, the stem cell transplant therapy is allogenic stem cell transplant therapy. In another embodiment, the stem cell transplant therapy is autologous stem cell transplant therapy. Autologous stem cell transplantation is preferably used for patients under the age of 65 years who do not have substantial heart, lung, renal or liver dysfunction. Autologous stem cell transplantation can be considered with a reduced-intensity conditioning regimen for older patients or those with coexisting conditions according to risk factor profile. Reduced-intensity conditioning regimen includes: reversible myelosuppression (usually within about 28 days) without stem cell support, mixed chimerism in a proportion of patients at the time of first assessment, and/or low rates of non-hematologic toxicity.

In another embodiment, the ezatiostat is administered as monotherapy.

In another embodiment, the therapeutically effective amount of said composition preferably provides for ezatiostat is a daily amount of about 500 mg-about 6,000 mg, or about 1,000 mg-about 5,000 mg, or about 1,500 mg-about 4,000 mg, about 1,000 mg-about 2,000 mg, about 2,000 mg-about 3,000 mg, about 3,000 mg-about 4,000 mg, about 4,000 mg-about 5,000 mg, or about 5,000 mg-about 6,000 mg. In a preferred embodiment, the ezatiostat is administered twice daily or BID, still more preferably in equal doses. In another embodiment, the composition is administered orally or parenterally. In another embodiment, the composition is administered as a solid dosage form, such as tablet forms described in U.S. Patent Application Publication US2011/0300215, which is incorporated by reference is its entirety. In another embodiment, the ezatiostat is present in the composition as substantially pure ezatiostat hydrochloride ansolvate, in particular, crystalline form D, described in U.S. Patent Application Publication 2011/0301088, which is incorporated by reference is its entirety. Substantially pure ezatiostat hydrochloride crystalline form D refers to a polymorphic form that contains about 90% or more, about 95% or more, or about 99% or more of the polymorphic form D. In another embodiment, the composition is administered from once every week to once every day.

In another aspect, the present invention provides a composition comprising an effective amount of ezatiostat to treat multiple myeloma and an effective amount of an additional drug to treat multiple myeloma. The effective amount of various agents suitable for treating multiple myeloma, administered alone or together with one or more agents, are well known to the skilled artisan and can be used in accordance to the present compositions. In a preferred embodiment, the additional drug is lenalidomide. In one embodiment, the composition further comprises a pharmaceutically acceptable excipient. A variety of such excipients suitable for compositions useful for oral and parenteral administration are well known to the skilled artisan.

The invention having been described in summary and in detail, is illustrated and not limited by the example below.

### EXAMPLE

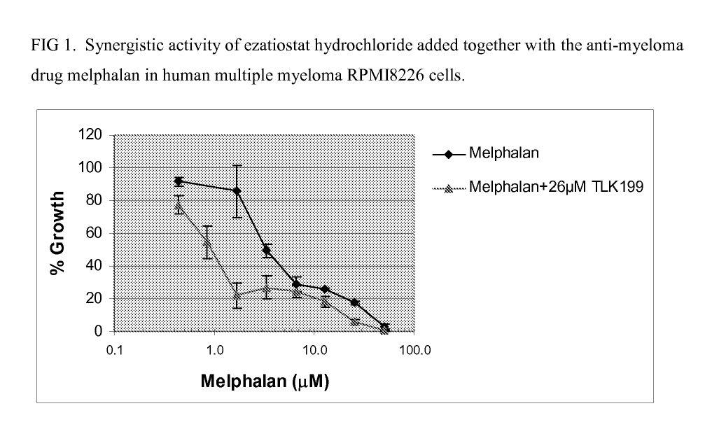

This example demonstrates the effect of ezatiostat hydrochloride, a pharmaceutically acceptable salt of ezatiostat, alone, and when added with another anti-myeloma drug, melphalan, on multiple myeloma proliferation. The effect that ezatiostat has on multiple myeloma proliferation is tabulated below (Table 1). When added together with melphalan, ezatiostat hydrochloride demonstrated synergy with melphalan in inhibiting human RPMI8226 multiple myeloma cell proliferation (FIG. 1).

TABLE 1

| Cell Line | Description | IC₅₀ (uM) |
|---|---|---|
| U266B1 | Human multiple myeloma | 0.86 |
| RPMI-8226 | Human multiple myeloma | 33.0 |
| ARH77 | Human multiple myeloma | 2.23 |

While this invention has been described in conjunction with specific embodiments and examples, it will be apparent to a person of ordinary skill in the art, having regard to that skill and this disclosure, that equivalents of the specifically disclosed materials and processes will also be applicable to this invention; and such equivalents are intended to be included within the following claims.

What is claimed is:

1. A method of treating a multiple myeloma tumor, which method comprises contacting said tumor with an effective amount of ezatiostat.

2. A method of killing a multiple myeloma cell comprising contacting the multiple myeloma cells with an effective amount of ezatiostat.

3. The method of claim 1 or 2, further comprising contacting one or more additional drugs to treat multiple myeloma.

4. A method of treating multiple myeloma in an afflicted patient which method comprises administering to said patient a therapeutically effective amount of a composition comprising ezatiostat.

5. The method of claim 4, wherein the therapeutically effective amount of said composition is administered as part of a first line (or induction) therapy.

6. The method of claim 4, wherein said composition is administered in combination with one or more additional drugs to treat multiple myeloma.

7. The method of claim 4, wherein therapeutically effective amount of said composition is administered as part of a salvage therapy in treating patients wherein the multiple myeloma has become refractory to other drugs.

8. The method of claim 7, wherein the multiple myeloma is refractory to one or more of bortezomib, cyclophosphamide, dexamethasone, doxorubicin, interferon-alpha, lenalidomide, melphalan, pegylated interferon-alpha, prednisone, thalidomide, and vincristine.

9. The method of claim 4, wherein the multiple myeloma is relapsed and/or refractory multiple myeloma.

10. The method of claim 9, wherein the relapsed multiple myeloma relapsed after treatment with one or more of bortezomib, cyclophosphamide, dexamethasone, doxorubicin, interferon-alpha, lenalidomide, melphalan, pegylated interferon-alpha, prednisone, thalidomide, and vincristine.

11. The method of claim 6, wherein said composition is administered as part of a salvage therapy in treating patients wherein the multiple myeloma has become refractory to other drugs.

12. The method of claim 11, wherein the multiple myeloma is refractory to one or more of bortezomib, cyclophosphamide, dexamethasone, doxorubicin, interferon-alpha, lenalidomide, melphalan, pegylated interferon-alpha, prednisone, thalidomide, and vincristine.

13. The method of claim 6, wherein the multiple myeloma is relapsed and/or refractory multiple myeloma.

14. The method of claim 13, wherein the relapsed multiple myeloma relapsed after treatment with one or more of bortezomib, cyclophosphamide, dexamethasone, doxorubicin, interferon-alpha, lenalidomide, melphalan, pegylated interferon-alpha, prednisone, thalidomide, and vincristine.

15. The method of claim 4, further comprises administering one or more additional therapies.

16. The method of claim 15, wherein the one or more additional therapies comprise allogenic stem cell transplant therapy.

17. The method of claim 15, wherein the one or more additional therapies comprise autologous stem cell transplant therapy.

18. The method of claim 1 or 4, wherein the ezatiostat is administered as monotherapy.

19. The method of claim 4, wherein the therapeutically effective amount of said composition provides for ezatiostat in a daily amount of about 500 mg-about 6,000 mg.

20. The method of claim 19, wherein the composition is administered orally or parenterally.

21. The method of claim 19, wherein the composition is administered as a solid dosage form.

22. The method of claim 21, wherein the ezatiostat is present in the composition as substantially pure ezatiostat hydrochloride crystalline form D.

23. The method of claim 4, wherein the composition is administered from once every week to once every day.

24. A composition comprising an effective amount of ezatiostat to treat multiple myeloma and an effective amount of another drug to treat multiple myeloma.
